(19) United States
(12) Patent Application Publication
(10) Pub. No.: US 2007/0275874 A1
(43) Pub. Date: Nov. 29, 2007

(54) Use of Leptin in Wound Healing

(75) Inventor: Maria Rocio Sierra-Honigmann, Thousand Oaks, CA (US)

Correspondence Address: DAVIS WRIGHT TREMAINE LLP, 865 FIGUEROA STREET, SUITE 2400, LOS ANGELES, CA, 90017-2566, US

(73) Assignee: YALE UNIVERSITY, New Haven, CT (US)

(21) Appl. No.: 11/573,769

(22) PCT Filed: Sep. 2, 2005
(86) PCT No.: PCT/US05/31455

§ 371 Date: Aug. 16, 2007

Related U.S. Application Data
(60) Provisional application No. 60/607,115, filed on Sep. 3, 2004.

(51) Int. Cl. A61K 38/17; A61P 17/02

(52) U.S. Cl. 514/2; 435/40.52

(57) ABSTRACT

As described herein, leptin treatment significantly increased wound contraction and epithelial regeneration while reducing granulation tissue and wound area, consistent with a healing augmentation effect. Specifically, in leptin-treated wounds, the inventor found increased expression of smooth muscle-actin (-SMA) and collagens I, III and IV. Taken together, the inventor's results indicate that a major functional theme for the accelerated wound healing action of leptin consists of the acute, local induction of genes whose expression are critical for repair and contraction. Thus, the invention relates to methods and compositions for the promotion and/or acceleration of wound repair, re-epithelialization, wound contraction and decrease of granulation tissue by administering leptin to the subject, as well as methods for studying this process.

### FIELD OF INVENTION

The present invention relates to the promotion and/or acceleration of wound repair by administering leptin to the subject.

### BACKGROUND OF THE INVENTION

Leptin

Leptin is produced from the obese (ob) gene and binds to the ob receptors (Ob-R). The ob gene is expressed in various tissues such as placenta, ovaries, muscle and adipose tissue. Leptin is produced in the adipocyte and in ovaries, and is a circulating 16 kDa protein (G. A. Bray, (1996) Lancet 348: 140; C. Liu et al., (1997) Endocrinology 138: 3548). Defective production of leptin results in gross obesity and type 2 diabetes in the obese (ob/ob) mouse. In humans, the leptin protein levels have been correlated to the percentage of body fat and is elevated in obese patients (R. V. Considine et al., (1996) N. Engl. J. Med. 334: 292). Defects in the leptin receptor, Ob-Rb, produce a syndrome in the mutant diabetic db/db mouse that is phenotypically identical to that observed in the ob/ob mouse. In addition to obesity, leptin is also believed to modulate estrogen expression and the fat stores needed for reproduction purposes. Other potential roles for leptin include regulation of hemopoiesis and macrophage function (T. Gainforth et al., (1996) Proc. Nat'l Acad. Sci. USA 93: 14564).

Leptin has been detected in the plasma of normal individuals and individuals receiving hemodialysis and in renal transplant patients, in placental tissue from pregnant women, and in cord blood of newborns (Respectively, J. K. Howard et al., (1997) Clin. Sci. 93: 119; S. G. Hassink et al., (1997) Pediatrics 100: 123). It has been suggested that leptin concentrations in newborns cannot be explained by adiposity alone. In women, leptin deficiency has been postulated to be involved with delayed puberty, menstrual disturbances and anorexia nervosa (M. Schwartz et al., (1997) N. Engl. J. Med. 336: 1802). Leptin is also believed to regulate lipid metabolism, glucose uptake, β-cell function, gonadotropin secretion, sympathetic tone, ovarian function and thermogenesis.

Glucocorticoids and insulin increase leptin production. Administration of leptin reduces food intake, decreases insulin concentrations, and lowers blood glucose concentrations in the ob/ob mouse, but not in the db/db mouse (G. A. Bray, (1996) Lancet 348: 140).

Leptin is a 16-kD protein closely related to the IL-6 cytokine family with direct biological effects on the hypothalamus, including appetite regulation and energy balance (B. E. Barton, (2001) Immunol. Res. 23: 41; J. L. Halaas et al., (1995) Science 269: 543). This paradigm of leptin action in the central nervous system (CNS) has been well described; however, it is more recently that additional non-CNS, peripheral effects of leptin have also been explored. Like other cytokine members of the IL-6 family, leptin has multiple pleiotropic effects. For example, it has been demonstrated that leptin can regulate islet β cell function, cellular immunity, monocyte and platelet activation, reproductive function and bone morphogenesis and angiogenesis (Kieffer et al., (1997) Diabetes 46: 1087; Lord et al., (1998) Nature 394; 897; Nakata et al., (1999) Diabetes 48: 426; Santos-Alvarez et al., (1999) Cell Immunol 194: 6.) Naturally occurring mutations in the mouse produce leptin- or leptin receptor (OB-Rb)-deficient states, giving rise to the Lep^{ob} (ob/ob) and Lepr^{ob} (db/db) mouse strains, respectively. As these animals characteristically develop morbid obesity and insulin resistance, they also exhibit a severely impaired wound healing phenotype. Thus, both Lep^{ob} and Lepr^{db} mouse strains have been widely used as models of pathological wound healing (H. D. Beer et al., (1997) J. Invest. Dermatol. 109: 132; D. G. Greenhalgh et al., (1990) Am. J. Pathol. 136: 1235; R. Tsuboi et al., (1992) J. Dermatol. 19: 673; W. H. Goodson et al., (1986) Diabetes 35: 491) In this regard, recent studies have shown that leptin treatment of wounds in Lep^{ob} mice reverses their healing impairment, accelerates wound closure and improves re-epithelialization (B. D. Ring et al., (2000) Endocrinology 141: 446; B. Stallmeyer et al., (2001) J Invest Dermatol 117: 98; Although these observations illustrate wound enhancement effects of leptin by macroscopic parameters of healing-focusing primarily on reversal of the healing impairment in Lep^{ob} mice-all possible pharmacological action of leptin to promote wound repair in normal animals has not been completely explored.

The Leptin Receptor

The leptin receptor belongs to the cytokine superfamily of receptors. Several forms of the leptin receptor are expressed in humans and rodents (G. A. Bray, (1996) Lancet 348: 140). The short form (Ob-R(S)), considered to have limited signaling capability, is detected in many organs and has 5 identified isoforms, Ob-Ra, Ob-Rc, Ob-Rd, Ob-Re, and r-Ob-Rf (M. Y. Wang et al., (1996) FEBS Letters 392: 87). Ob-R(S) has been identified in the choroid plexus and may be involved in the transport of leptin across the blood-brain barrier (J. Girard, (1997) Diabetes Metabol. 23S: 16).

It is the long form of the leptin receptor which is believed to mediate the biological effects of the leptin protein (L. A. Campfield et al., (1996) Horm. Metab. Res. 28: 619). In contrast to the short form of the leptin receptor, Ob-R long form (Ob-R (L) also known as Ob-Rb) predominates in the hypothalamus and cerebellum (A. Savioz et al., (1997) Neuroreport 8: 3123; J. G. Mercer et al.; (1996) FEBS Letters 387: 113).

Ob-R (L) has also been detected at low concentrations in peripheral tissues (Y. Wang et al., (1997) J. Biol. Chem. 272: 16216), such as the brain (A. Heritier et al., (1997) Neurosci. Res. Commun. 21: 113), spleen, testes, kidney, liver, lung, adrenal (N. Hoggard et al., (1997) Biochem. Biophvs. Res. Commun. 232: 383), and hematopoietic tissues (A. A. Mikhail et al., (1997) Blood 89: 1507). Ob-R (L) has also been observed in the placenta, fetal cartilage/bone, and hair follicles, and therefore is believed to play a role in development (N. Hoggard et al., (1997) Proc. Nat'l Acad. Sci. USA'94: 11073).

Ob-R (L) has been demonstrated to transduce intracellular signaling in a manner analogous to that observed for interleukin (IL)-6 type-cytokine receptors. Ob-R (L) transmits its information via the Janus kinases (JAK), specifically Jak2 (N. Ghilardi et al., (1997) Mol. Endocrinol. 11: 393), which subsequently phosphorylate transcription factors of the STAT3 family (J. Girard (1997)).

Leptin sensitizing compounds have also been disclosed. See, for example, PCT Publication No. 98/02159.

Angiogenesis

Angiogenesis refers to the growth of new blood vessels, or “neovascularization,” and involves the growth of new blood vessels of relatively small caliber composed of endothelial cells. Angiogenesis is an integral part of many important biological processes including cancer cell proliferation solid tumor formation, inflammation, wound healing, repair of injured ischemic tissue, myocardial revascularization and remodeling, ovarian follicle maturation, menstrual cycle, and fetal development. New blood vessel formation is required for the development of any new tissue, whether normal or pathological, and thus represents a potential control point in regulating many disease states, as well as a therapeutic opportunity to encourage growth of normal tissue and “normal” angiogenesis.

The complete process for angiogenesis is not entirely understood, but it is known to involve the endothelial cells of the capillaries in the following ways: (1) the attachment between the endothelial cells and the surrounding extracellular matrix (ECM) is altered, presumably mediated by proteases and glycosidases, which permit the destruction of the basement membrane surrounding the microvascular endothelial cells, thus allowing the endothelial cells to extend cytoplasmic buds in the direction of chemotactic factors; (2) there is a “chemotactic process” of migration of the endothelial cells toward the tissue to be vascularized; and (3) there is a “mitogenesis process” (e.g., proliferation of the endothelial cells to provide additional cells for new vessels).

Each of these angiogenic activities can be measured independently utilizing in vitro endothelial cell cultures.

A number of factors are known to stimulate angiogenesis. Many of these are peptide factors, and the most notable of these are the fibroblast growth factors (FGF), both acidic (aFGF) and basic (bFGF), which can be isolated from a variety of tissues including brain, pituitary and cartilage. FGFs are characterized by their heparin-binding properties. Heparin is a powerful anticoagulant agent normally found in minute amounts in the circulatory system. Other factors known to show angiogenic-stimulating activity, include but are not limited to: vascular endothelium growth factor (VEGF), angiopoietin I and II, prostaglandins E1 and E2 (B. M. Spiegelman et al., 1992), ceruloplasmin, monocyte derived monocytoangiotropin, placental angiogenic factor, glioma-derived endothelial cell growth factor, and a heparin-binding growth factor from adenocarcinoma of the kidney that is immunologically related to bFGF (R. B. Whitman et al., (1995) U.S. Pat. No. 5,470,831). Platelet-derived endothelial cell growth factor (PD-ECGF) does not stimulate proliferation of fibroblasts in contrast to the FGFs, but has demonstrated in vitro angiogenic activity (see C. H. Heldin et al., (1993) U.S. Pat. No. 5,227,302).

Factors are also known that are capable of inhibiting endothelial cell growth in vitro. One of the most extensively studied inhibitors of endothelial cell growth is protamine, which is found only in sperm. Platelet factor 4 (PF4) and major basic protein also have been demonstrated to have inhibitory effects on angiogenesis (T. Maione, (1992) U.S. Pat. No. 5,112,946). Oncostatin A, which is similar to native PF4, has also been implicated as effecting the growth of tumors and therefore may act as an angiogenesis inhibitor (T. Maione, 1992). Antibodies have also been created possessing anti-angiogenic activity (see for example, C. R. Parish (1997) U.S. Pat. No. 5,677,181).

Gene therapy has also been contemplated as a means of promoting or inhibiting angiogenesis (T. J. Wickhane et al., (1996) J. Virol. 70: 6831).

Wound Healing and Repair of Tissue after Ischemic Injury

Wounds are internal or external bodily injuries or lesions caused by physical means, such as mechanical, chemical, bacterial, or thermal means, which disrupt the normal continuity of structures. Such bodily injuries include contusions, wounds in which the skin is unbroken, incisions, wounds in which the skin is broken by a cutting instrument, and lacerations, wounds in which the skin is broken by a dull or blunt instrument. Wounds may be caused by accidents or by surgical procedures. Additional examples include, but are not limited to, bone repair, burns, post-infarction in myocardial injury, gastric ulcers and other ulcers of the gastrointestinal tract. Wounds may be caused by accidents or by surgical procedures.

Wound healing consists of a series of processes whereby injured tissue is repaired, specialized tissue is regenerated, and new tissue is reorganized. Wound healing is usually divided into three phases: the inflammatory phase, the proliferative phase, and the remodeling phase. Fibronectin has been reported to be involved in each stage of the wound healing process, particularly by creating a scaffold to which the invading cells can adhere. Initially, many mediators, such as fibronectin and fibrinogen, are released to the wound site. Thereafter, angiogenesis and re-epithelialization take place (A. Beauliu (1997) U.S. Pat. No. 5,641,483). Repair of injured tissue due to ischemia is a form of wound healing which requires extensive remodeling and re-vascularization. An infarct is, by definition, and area of tissue ischemic necrosis caused by occlusion of local blood circulation. The resulting necrotic lesion leaves the affected tissue deprived of oxygen and nutrients. In the heart, obstruction of coronary circulation in particular, results in myocardial infarction. As the ischemic myocardium undergoes rapid oxygen starvation, the hypoxic microenvironment of the infected cardiac muscle induces the synthesis of angiogenic factors to attempt re-vascularization. For example vascular endothelium growth factor (VEGF) is known to be produced in the areas of the myocardium that have undergone an infarction (Ref. Similarly, ischemic injured tissue outside the heart also produces various angiogenic factors.

Adult wound healing in response to injury results in restoration of tissue continuity (Adzick N. S. et al. (eds), in Fetal Wound Healing, Elsevier, N.Y. 1992, Chapters 13, 12, 13 and references cited therein). While some amphibians heal by regeneration, adult mammalian tissue repair involves a complex series of biochemical events that ultimately ends in scar formation. The events occurring during wound repair resemble the process of development, including synthesis, degradation and re-synthesis of the ECM (Smith L. T. et al., (1982) J. Invest. Dermatol. 79: 935; Blanck C. E. et al., (1987) J. Cell. Biol. 105: 139 (A)). The ECM contains several macromolecules, including collagen, fibronectin, fibrin, proteoglycans, and elastin. When the injury involves the dermis, repair also entails the removal of cellular debris, and the laying down of a new ECM over which epidermal continuity can be reestablished. This process of repair and dermal matrix reorganization is manifested as scar formation and maturation.

Manipulation of the wound healing environment by the application of extrinsic growth factors such as fibroblast growth factor (FGF) and transforming growth factory (TGFβ) (T. A. Mustoe et al., (1987) Science 237: 1333; S. M. Seyedin et al, (1986) J. Biol. Chem. 261: 5693) can influence the early stages of scar formation. During tissue repair, TGFβ modulates the inflammatory response as a potent chemoattractant for fibroblasts, macrophages, neutrophils and T lymphocytes. TGFβ can also upregulate cell surface expression of the integrins that act as receptors for fibronectin, collagen, laminin, and vitronectin thereby influencing cell adhesion and migration. TGFβ enhances the epithelial covering of exposed dermis and increases tensile strength in incision wounds.

See J. W. Siebert et al., (1997) U.S. Pat. No. 5,591,716) for additional discussion of growth factors that are involved in the process of wound healing and scarring.

There is a need in the art for improvements in wound healing technology and methods for studying the same.

### SUMMARY OF THE INVENTION

The following embodiments and aspects thereof are described and illustrated in conjunction with compositions and methods which are meant to be exemplary and illustrative, not limiting in scope.

This invention relates to a method of modulating angiogenesis, repair of ischemic tissue and wound healing using leptin and leptin receptors. Leptin or its analogs or its specific inhibitors or other agents that modulate the leptin receptor or agents that may induce leptin or leptin receptor synthesis can be administered to the subject in an amount effective to produce an angiogenic response.

Other reagents contemplated for use in modulating angiogenesis include leptin homologues, angiogenic peptide fragments of leptin, idiotypic antibodies that bind to the leptin binding site on the leptin receptor, leptin sensitizers, and an angiogenesis-inducing compound released by a tumor.

Another aspect of the invention relates to the use of one or more agents that regulate angiogenesis in combination with compounds which modulate leptin activity, leptin receptor activity and/or leptin receptor ligand activity. The other agents to be used in combination include VEGF, FGF, PDGF, TGF-β, angiopoietin, TNF and leptin sensitizers.

Methods of treating undesired angiogenesis in a subject are also contemplated. One method comprises the step of administering to the subject an effective amount of an agent that modulates leptin expression or leptin receptor activity sufficient to modulate the undesired angiogenesis.

Another aspect of this invention relates to antibodies that bind to the leptin receptor, wherein the binding of the antibody to the receptor modulates leptin receptor-mediated response by the cell to an angiogenesis-inducing stimulus.

This invention also discloses methods of promoting and/or accelerating wound healing and repair of ischemic tissue (which are conditions mediated by angiogenesis). Embodiments of the present invention include methods to promote and/or accelerate wound repair in a vertebrate specie, including providing a composition comprising a quantity of leptin and/or its analogs and administering a therapeutically effective amount of the composition to the vertebrate specie. Other embodiments include methods for promoting and/or accelerating wound contraction. Additional embodiments include methods for promoting and/or accelerating re-epitheliazation. Further embodiments include methods to decrease granulation tissue in a wound. In one embodiment of the present invention, the vertebrate specie is a mammal. In another embodiment of the present invention, the mammal is a human.

One aspect of the invention includes compositions such as a wound dressing comprising at least leptin and a suitable carrier. Other wound healing compositions contemplated include a topical composition comprising at least one agent that modulates a response in a subject to an angiogenesis-inducing stimulus, comprising an effective amount of an agent that modulates leptin or leptin receptor mediated angiogenic response to that stimulus, together with a pharmaceutically acceptable carrier. In one embodiment, the agent is leptin. In one embodiment, the leptin receptor contemplated is the long form, however other isoforms of the leptin receptor may also be used.

Further embodiments include methods for treating or modulating wound healing in vertebrates, such as humans, utilizing pharmaceutical compositions. One method for promoting the formation, maintenance or repair of tissue, comprises the step of administering, to a subject in need thereof, an effective amount of an agent that induces a leptin or leptin receptor-mediated angiogenic response in the subject. This response can affect vascular cells such as endothelial cells or vascular smooth muscle cells. This can also affect epithelial cells, granulation tissue and contraction of the wound. In one embodiment, the administration of agents is local, although systemic administration is also contemplated. These agents can be used in combination with other angiogenic agents such as VEGF, FGF, PDGF and leptin sensitizers. One example would be the administration of leptin and VEGF to enhance wound healing. Other agents to be used in combination with leptin include TGF-P, angiopoietin, and TNF. Pharmaceutical compositions disclosed for the treatment of skin wounds are based on a pharmaceutical composition comprising at least one agent that modulates leptin or leptin receptor activities and/or their synthesis or degradation. In use, such compositions may be applied directly, and may be applied first to a dressing material and then the impregnated dressing material is applied to wounded or traumatized skin. The dressing material may also include at least one additive selected from the group comprising: keratolytics, surfactants, counterirritants, humectants, antiseptics, lubricants, astringents, emulsifiers, wetting agents, wound healing agents, adhesion/coating protectants, vasoconstrictors, antichlolinergics, corticosteroids, anesthetics and anti-inflammatory agents.

Various embodiments of the present invention relate to methods and compositions for the treatment of wounds in vertebrate species, for example, mammal, human, bovine, and avian.

In further embodiments, the present invention includes compounds that affect the leptin receptor to promote and/or accelerate wound repair.

In various embodiments of the present invention, the composition may include additional active ingredients to promote and/or accelerate wound repair.

Another embodiment of the present invention includes a kit, including a composition comprising a quantity of leptin, and instructions for its use to promote wound repair in a mammal.

Further embodiments of the present invention include methods and techniques for the study and evaluation of wound healing and/or repair using quantitative micromorphometric analysis.

Other features and advantages of the invention will become apparent from the following detailed description, taken in conjunction with the accompanying drawings, which illustrate, by way of example, various features of embodiments of the invention.

### BRIEF DESCRIPTION OF FIGURES

Exemplary embodiments are illustrated in referenced figures. It is intended that the embodiments and figures disclosed herein are to be considered illustrative rather than restrictive.

FIG. 1 illustrates a mouse model for studying the effects of leptin on wound healing by micromorphometry. (A) Diagram outlining the different steps of the wound model and the micromorphometric analysis. Each mouse was subjected to bilateral full-thickness incisional wounds, 8-mm in length. After 72 hours, wounds were bisected and processed for histology. Digital images obtained from the hematoxylin and eosin (H&E) slides were analyzed with an imaging software program for several parameters of wound healing. (B) Paraffin section obtained from the bisected lesion and stained with H&E shows the typical staining pattern of a wound image (i) and (ii); (iii) wound contraction (an estimate of wound closure) was measured as dermal border distance indicated by arrows (DBd); (iv) measured wound closure is illustrated as epithelial border distance (EBd) comprising the distance between discernible epithelial tongues on both sides of the wound section; (v) granulation tissue area and (vi) wound area measurements are shown enclosed by red dashed lines (GTa: granulation tissue area, Wa: wound area). (C) Comparison of the computer-assisted measurements performed by two investigators on 60 digital micrographs of wounds selected at random and blinded to the individuals performing the observations. The investigators were instructed on the measuring technique and given the same set of micrographs. Each individual performed the measurements independently, which were recorded and compared side-by-side. The results are presented as average ±S.E. [* Investigator 1 (E.D.) and ** Investigator 2 (S.T.C.)]. Area measurements are expressed as square millimeters (mm²) and linear distance as millimeters (mm).

FIG. 2 illustrates histological and micromorphometric assessment of control and leptin-treated incision wounds. Histological sections of wounds obtained and processed as described in FIG. 1. A single treatment was applied immediately after wounding and the tissue was collected after 72 hours. Representative photomicrograph of saline and leptin-treated wounds depicting typical healing patterns. (A) Saline-treated wound showing the normal features of a wound in the process of healing with incomplete epithelium closure and discrete contraction, abundant granulation tissue and large overall wound area (100×). (B) Higher magnification (400×) showing details of the wound border with hyperproliferative epithelium tongue. (C) Leptin-treated wound showing accelerated healing, greater degree of contraction, complete re-epithelialization, and decreased granulation tissue, infiltrate and wound area (100×). (D) Higher magnification (400×) shows full regeneration of the epithelial layer across the wound (E, epithelium; D, dermis; GT, granulation tissue; *denotes areas shown at higher magnification in B and D). (E) Computer-assisted micromorphometric measurements performed on histological sections of control (S, solid bars) and leptin-treated wounds (L, hatched bars) expressed as the reciprocal value of the linear distance between dermal borders (wound contraction), or between epithelial tongues of the neoepithelium (wound re-epithelialization or closure). (F) Comparative change in granulation tissue and overall wound area after treatment with saline (S, solid bars), or a single dose of leptin (10 μg; L, hatched bars) at the time of wounding. (average ±S.E.M., p<0.01 for all parameters; n=22 for each group).

FIG. 3 illustrates comparative time course of healing progression of control and leptin treated wounds. (A) Histological sections of wounds obtained at various times. A single treatment of leptin or saline vehicle was applied immediately after wounding and the tissue was collected after euthanasia at the indicated times. Control wounds showing the normal progression of healing from the early inflammatory phase on day 1, through the granulation tissue (*) formation phase and epithelial advance from the wound borders (arrows) on days 2 and 3 until day 5, when closure of the epidermis is completed with remaining granulation tissue, infiltrate and scar remodeling morphology (**). In contrast, day-1 leptin-treated wounds display characteristics similar to those observed on day-3 controls, with closure by day 3 and signs of scar remodeling on day 5 (200×). (B) Macroscopic appearance of excision wounds at 24 hours and on day 7. The macroscopic aspect of control and leptin-treated wounds are almost indistinguishable after 24 hours, but quite different on day 7. (C) Morphometric assessment of granulation tissue areas in control and leptin-treated wounds. Leptin treatment decreases the overall area of granulation tissue when compared to control wounds. However, discernable granulation tissue is already apparent on days 1 and 2, in contrast to the controls wounds where maximal granulation tissue is detected by day 3 (saline, hatched bars; leptin, solid bars). Results are expressed as average ±S.E.M., (p<0.01; n=10 for each group).

FIG. 4 illustrates dose-dependent response of incision wounds to topical treatment with leptin. Micromorphometric assessment of healing progression as a function of increasing doses of leptin. Measurements were done on day-3 wounds, according to the method described earlier (FIG. 1). Each wound received the indicated dose of leptin at the time of wounding. (A) Wound contraction; (B) wound epithelialization; (C) granulation tissue area; (D) Wound area. Saline, hatched bars; leptin, solid bars. Results are expressed as average ±S.E.M. (p<0.01; n=11 for each group).

FIG. 5 illustrates presence of myofibroblasts and increased smooth muscle α-actin mRNA expression on day-3 leptin treated incision wounds. Immunohistochemical detection of smooth muscle α-actin was performed as described in Detailed Description of the Invention on (A) control wounds and (B) leptin-treated wounds (10 μg/wound). (C) High magnification (400×) of the region shown by first arrow of panel B. (D) High magnification (400×) of the region shown by second arrow of panel B. (E) Smooth muscle α-actin mRNA expression in saline control and leptin-treated wounds (10 μg/wound).

FIG. 6 illustrates changes in collagen expression on day-5 leptin treated incision wounds. (A) Picrosirius Red staining of saline control and leptin-treated incision wounds depicting appearance of collagen fibrils on selected areas of each wound including the scar tissue proper forming on the edge of the wound (*), a more loosely organized matrix replacing the area of granulation tissue (**), and matrix on the wound scab (***). Bar length is 200 μm for top two panels and 50 μm for lower six panels. (B) Time course of mRNA expression for collagen α1(I), α1(III) and α1(IV) in saline-treated controls (empty symbols) and leptin-treated wounds (filled symbols).

### DETAILED DESCRIPTION OF THE INVENTION

All references cited herein are incorporated by reference in their entirety as though fully set forth. Specifically, the International Application Publication No. WO 99/59614, “Modulation of Angiogenesis and Wound Healing,” is incorporated by reference in its entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” include, but are in no way limited to, lessening or alleviating the severity of a wound or its complications, merely preventing or inhibiting it from worsening, healing the wound, reversing the progression of the wound, ameliorating the wound, restoring tissue continuity, repairing of injured tissue, decreasing granulation tissue area, promoting and/or accelerating re-epithelialization, generating specialized tissue, reorganizing of new tissue, or a therapeutic effort to effect any of the aforementioned, even if such therapeutic effort is ultimately unsuccessful.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be including within the scope of this term.

“Therapeutically effective amount” as used herein refers to that amount which is capable of achieving beneficial results in a patient with a wound. A therapeutically effective amount can be determined on an individual basis and will be based, at least in part, on consideration of the physiological characteristics of the mammal, the type of delivery system or therapeutic technique used and the time of administration relative to the progression of the wound.

“Treatment” and “treating,” as used herein refer to both therapeutic treatment and prophylactic or preventative measures, wherein the object is to promote, enhance and/or accelerate the wound repair, even if the treatment is ultimately unsuccessful.

“Leptin” as used herein refers to the leptin protein, a product of the ob gene, and its allelic variants and homologues as found (or as is believed to be found) in all vertebrate species, including human, bovine, avian, etc. Leptin encoding nucleic acid molecules include allelic variants, mutants and nucleic acids that encode biologically active variants. The “biologically active variants” are those leptin variants that can induce angiogenic activity and/or enhance wound healing. Leptin nucleic acid molecules also encompass cDNAs, RNAs, recombinant RNAs and DNAs, and antisense molecules.

“Leptin receptor” as used herein includes the long form, Ob-R (L), and the short form, Ob-R(S) or Ob-Rb, as well as other leptin receptor isoforms. “Leptin receptor” also includes allelic variants and homologues as found in most or all vertebrate species, including human, bovine, avian, etc. Leptin receptor encoding nucleic acid molecules include allelic variants, mutants and nucleic acids that encode biologically active variants of the leptin receptor. The “biologically active variants” are those leptin receptor variants that are involved in the leptin-mediated induction of angiogenic activity and/or leptin mediated enhancement of wound healing. Leptin receptor nucleic acid molecules also encompass cDNAs, RNAs, recombinant RNAs and DNAs, and antisense molecules.

“Polypeptide fragments” and “peptide fragments” as used herein refer to portions of leptin and the leptin receptor capable of modulating angiogenesis, wound healing, and/or repair of ischemic tissue activity. Such polypeptides, and derivatives or analogs thereof, as contemplated by the present invention are those that have the ability to inhibit angiogenesis, wound healing and/or repair of ischemic tissue, or to promote angiogenesis, wound healing and/or repair of ischemic tissue by affecting leptin receptor activity, leptin activity and/or leptin receptor ligand activity. These polypeptides and peptides encompass derivatives, analogs and peptidomimetics (i.e., molecules having some structural and functional characteristic in common with peptides, but that do not contain peptide bonds). One embodiment includes leptin and fragments thereof that bind to the leptin receptor. Another embodiment encompassed by “leptin polypeptides” or “leptin receptor polypeptides” are fragments of these peptides comprising at least about 2, 3, 5, 10, 15, 20, 25, 30 or 50 consecutive amino acid residues.

“Wounds” are internal or external bodily injuries or lesions caused by physical means, such as mechanical, chemical, bacterial, or thermal means, which disrupt the normal continuity of structures. Such bodily injuries may include, but are in no way limited to contusions; wounds in which the skin is unbroken, incisions, wounds in which the skin is broken by a cutting instrument, and lacerations, wounds in which the skin is broken by a dull or blunt instrument. Additional examples include, but are not limited to, bone repair, burns, post-infarction in myocardial injury, gastric ulcers and other ulcers of the gastrointestinal tract. Wounds may be caused by accidents or by surgical procedures.

“Granulation tissue” as used herein refers the highly vascularized tissue that replaces the initial fibrin clot in a wound. Vascularization is by ingrowth of capillary endothelium from the surrounding vasculature. The tissue is also rich in fibroblasts (that will eventually produce the fibrous tissue) and leucocytes.

“Epithelium” as used herein refers to outside layer of cells that covers all the free, open surfaces of the body including the skin, and mucous membranes that communicate with the outside of the body.

“Dermis” as used herein refers to the lower or inner layer of the two main layers of cells that make up the skin. The dermis contains blood vessels, lymph vessels, hair follicles, and glands that produce sweat.

“Contraction” and “wound contraction” refer to a shortening or reduction of the size of the wound.

“Wound epithelialization” and “re-epithelialization” as used herein refer to the process of becoming covered with or converted to epithelium.

“Vertebrate specie” as used herein refers to an animal of the subphylum, Vertebrata, comprising animals, such as mammals, birds, reptiles, amphibians, and fishes, with a segmented spinal column.

“Modulating” as, used herein means the ability to regulate a biological effect or process, such as repair of ischemic tissue, wound healing and/or angiogenesis. Modulation can occur by “inhibiting”, “blocking”, “down-regulating” or “depressing” leptin and/or leptin receptor-mediated activity. Modulation also encompasses instances wherein leptin or leptin receptor activity is “induced”, “up-regulated”, “increased”, “promoted”, or “enhanced”.

“Anti-angiogenic effect” as used herein means a morphological response that inhibits or blocks vascularization including neovascularization or revascularization. An “anti-angiogenic effect” is one wherein vascularization and associated morphological changes in vascular cells, such as endothelial cells and vascular smooth muscle cells, does not occur or is inhibited. The terms “angiogenic” and “angiogenesis” refer to revascularization or neovascularization of tissue. Such neovascularization can result from the process of wound healing, repair of ischemic tissue or tissue growth. An “angiogenic effect” can be one wherein vascularization occurs or morphological changes associated with angiogenesis are observed in vascular cells such as endothelial cells (“EC”) and vascular smooth muscle cells.

“Agonists” include, but are not limited to, those agents, compounds, compositions, which when administered can up regulate (increase, promote or otherwise elevate the level of) angiogenesis and/or wound healing by promoting leptin activity, leptin receptor activity, leptin/leptin receptor interaction, or a combination thereof.

“Antagonists” include, but are not limited to, those agents, compounds, compositions, etc. which when administered cause the down regulation (inhibition, prevention, reduction, etc.) of angiogenesis, wound healing and/or repair of ischemic tissue by inhibiting leptin activity, leptin receptor activity, leptin/leptin receptor interaction, or a combination thereof.

“Isolated” DNA, RNA, peptides, polypeptides, or proteins are DNA, RNA, peptides polypeptides or proteins that are isolated or purified relative to other DNA, RNA, peptides, polypeptides, or proteins in the source material. For example, “isolated DNA” that encodes leptin (which would include cDNA) refers to DNA purified relative to DNA which encodes polypeptides other than leptin.

Disease states and other conditions involving “angiogenic activity” include, but are not limited to myocardial conditions, trauma, tumors (benign and malignant) and tumor metastases, ischemia, tissue and graft transplantation, diabetic microangiopathy, neovascularization of adipose tissue and fat metabolism, revascularization of necrotic tissue, eye conditions (e.g., retinal neovascularization), growth of new hair and ovarian follicle maturation.

Disease states and other conditions involving “wound healing” include: scarring and scar formation, ischemia, burns, myocardial injury, enhancement of vascularization in microvascular transplants, enhancement of revascularization in necrotic tissue and tissue and graft transplants. Also contemplated is enhancement of wound healing in subject with poor wound healing, as in diabetic individuals. These conditions may be mediated by modulation of leptin, leptin receptor, and leptin receptor ligands activity.

“Vascular cells” include both “endothelial cells” (also referred to as “EC”) and “smooth muscle cells” and “vascular smooth muscle cells” (also referred to as “SMC”).

The inventor's findings as described herein suggest that leptin-based therapies may have clinical applications not only in wound healing and/or repair, but alsd in other instances with similar underlying pathophysiology. For instance, in diseases and conditions involving angiogenic activity, such as, but not limited to, myocardial conditions, ischemia, and tumors wherein the activity generally involves the endothelial cells of the capillaries whereby (1) the attachment between the endothelial cells and the surrounding extracellular matrix (ECM) is altered, presumably mediated by proteases and glycosidases, which permit the destruction of the basement membrane surrounding the microvascular endothelial cells, thus allowing the endothelial cells to extend cytoplasmic buds in the direction of chemotactic factors; (2) there is a “chemotactic process” of migration of the endothelial cells toward the tissue to be vascularized; and (3) there is a “mitogenesis process”. In these processes, the angiogenic activity may be promoted by leptin-based therapies and thus accelerate the treatment of these disease conditions. Alternatively, in appropriate instances, the angiogenic activity may be inhibited by leptin-based therapies and thus decelerate or halt the progression of these disease conditions.

Additionally, leptin's role in the possible modulation of discrete events such as recruitment of fibrocytes to the injured site, their differentiation into myofibroblasts within the wound bed, or changes in their contractile function may also be of significance in other disease conditions involving these changes. The possible autocrine and paracrine effects due to leptin may also aid treatment of other disease conditions.

One skilled in the art will readily recognize other conditions as which modulation of these pathophysiologic mechanisms would be desirable.

The invention includes methods and compositions for treating diseases and/or conditions mediated by angiogenesis, or conditions associated with repair of ischemic tissue or wound healing by utilizing reagents that modulate leptin and/or the leptin receptor, including but not limited to leptin.

Methods of Treating Diseases and Conditions

This section describes the diseases wherein reagents can be administered to a subject to enhance or inhibit angiogenesis, wound healing and/or repair of ischemic tissue. The subjects contemplated include all vertebrate species. Various embodiments include methods of treating diseases in mammals, and one method is the treatment of humans. The control of angiogenesis, wound healing and/or repair of ischemic tissue can alter the pathological damage associated with the disease or with abnormal angiogenesis. “Abnormal angiogenesis” can be an irregular or abnormal level of neovascularization (e.g., enhanced or depressed neovascularization).

The invention includes methods to promote and/or accelerate wound repair by providing a composition comprising a quantity of leptin and administering a therapeutically effective of the composition to a vertebrate specie, including mammal, human, bovine, avian, etc. In one embodiment of the present invention, the vertebrate specie is a mammal. In another embodiment of the present invention, the mammal is a human. Additional embodiments include treatment of veterinary animals, such as farm animals, domestic animals and laboratory animals. The leptin may be formulated into an appropriate pharmaceutical composition for use in connection with leptin delivery techniques as contemplated by alternate embodiments of the present invention.

Diseases Wherein Angiogenesis should be Inhibited

Angiogenesis should be inhibited in diseases or conditions in which it is desirable to block or inhibit neovascularization. In a broad view, the conditions and diseases where angiogenesis desirably may be inhibited include: scar formation, tumor metastasis and tumor growth, and tissue adhesions. More specifically, these conditions and diseases include ocular neovascular diseases (e.g., including diabetic retinopathy, diabetic microangiopathy, retinal neovascularization, retinopathy of prematurity, corneal graft rejection, neovascular glaucoma, and retrolental fibroplasia), other diseases associated with corneal neovascularization (e.g, include: epidemic keratoconjunctivitis, vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegeners sarcoidosis, Scleritis, Steven's Johnson disease, periphigoid radial keratotomy and corneal graft rejection), diseases associated with retinal/choroidal neovascularization (e.g., diabetic retinopathy, macular degeneration, sickle cell anemia, sarcoid syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, Bests disease, myopia, optic pits, Stargarts disease, pars planitis, chronic retinal detachment, hyperviscosity syndromes, toxoplasmosis, trauma and post-laser complications), diseases associated with rubeosis (neovascularization of the angle), regulation of neovascularization or active angiogenesis in adipose tissue, and diseases caused by the abnormal proliferation of fibrovascular or fibrous tissue including all forms of proliferative vitreoretinopathy.

Chronic inflammation may also involve pathological angiogenesis. Diseases with chronic inflammatory conditions considered for treatment using the methods of the present invention include: ulcerative colitis, Crohn's disease, rheumatoid arthritis, and Bartonellosis.

Neovascularization also occurs in both benign and malignant tumors, and the vascular endothelial cells and vascular smooth muscle cells in the vicinity of tumors, particularly those cells within the range of tumor-produced angiogenic factors, therefore correspondingly are also contemplated as targets for therapy. Examples of tumor diseases that are contemplated as being appropriate for treatment by the methods of the present invention include, but are not limited to: systemic forms of hemangiomas, hemangiomatosis, Osler-Weber-Rendu diseases, hereditary hemorrhagic telangiectasia, rhabdomyosarcomas, retinoblastomas, Ewing sarcomas, neuroblastomas adenocarcinomas and osteosarcomas.

In wound healing, excessive repair or fibroplasia can have detrimental side effects on surgical procedures and may be caused or exacerbated by angiogenesis.

Correspondingly, these therapies also may be utilized to inhibit undesired scar formation.

Methods of Treating Diseases and Conditions by Up-Regulating Angiogenesis

In other diseases, angiogenic activity may need to be enhanced to promote neovascularization and/or wound healing. Diseases and conditions contemplated for said treatment include: myocardial ischemic conditions (e.g., myocardial infarction, revascularization of necrotic tissue, for example of the myocardium after an infarction or an angioplasty, angina, heart transplants, vascular grafts, and reopening vessels to improve vascularization, perfusion, collagenization and organization of said lesions), ovarian follicle maturation (which may also require down regulation of angiogenesis), wound healing, and tissue and organ transplantations (e.g., enhancement of autologous or heterologous microvascular transplantation). Promotion of wound healing includes healing of incisions, bone repair, burn healing, post-infarction repair in myocardial injury, healing of gastric ulcers and other ulcers of the gastrointestinal tract and generally in promoting the formation, maintenance and repair of tissue. Neovascularization of grafted or transplanted tissue is also contemplated, especially in subjects suffering from vascular insufficiency, such as diabetic patients.

Wound Healing

The dynamic process of wound healing is a well regulated sequence of events which, under normal circumstances, results in the successful repair of injured tissues.

First, a cutaneous wound that cuts through the epidermis and dermis (full thickness), is accompanied by blood vessel rupture. Rapidly, clot formation occurs providing a provisional matrix to cover the wound. The clot is a key component because it provides mechanical closure with fibrin and other matrix proteins, and it is the initial source of cytokines, growth factors and chemotactic agents released by platelet degranulation. This cocktail initiates the process of wound healing. Next, neutrophils move into the interstitum at the site of injury in response to bacterial products and other chemotactic agents. This is followed by macrophages that release chemical signals to attract fibroblasts. The resident and infiltrating fibroblasts secrete cytokines such as PDFG-BB and bFGF and begin to deposit a new extracellular matrix that will be an essential component of the scar tissue. Meanwhile, the process of reepithelialization begins on the borders of the wound where keratinocytes of the basal layer display new integrins to attach to a provisional matrix. The epidermal migration continues until a monolayer of keratinocytes covers the wound. Several known growth factors intervene in the reepithelialization of the skin (e.g., EGF, TGFa and KGF 1 and 2).

In the underlying dermis, the process of neovascularization is established in response to severed vessels and angiogenic factors produced locally. The role of the microvasculature in wound healing is essential for the repair to take place. After the interruption in the continuity of the microvasculature, endothelial cells need to dissolve their cell-cell attachments, migrate outside the vesssel into the extracellular matrix, undergo mitosis and finally reassociate in an orderly manner to form a network of capillaries necessary for the healing to proceed. It appears that VEGF secreted acutely by the keratinocytes is responsible in great part for the angiogenic response. Other angiogenic factors like basic fibroblast growth factor (bFGF) and transforming growth factor b (TGFb) are also present. The inventor believes that leptin is angiogenic, therefore, while not wishing to be bound by any particular theory, the inventor believes that leptin is involved in normal wound healing. Leptin, a protein produced in the underlying adipose tissue, may be present at relatively high concentrations because the dermal vasculature, both superficial and deep plexuses, derives from larger vessels that originate from the subcutaneous adipose layer.

Normal healing involves proliferation, migration, matrix synthesis and angiogenesis. An impairment at any of these complex phases will lead to complications in wound healing. In diseases of impaired neovascularization, such as diabetes, dermal wound healing is severely compromised. This often leads to nonhealing wounds and, ultimately, amputation. Recombinant protein therapy with leptin may augment angiogenesis and can be of great value in diabetes and other clinical situations where healing is impaired.

The present inventor observed that leptin plays a role in normal wound healing. Leptin is present at the wound site a few hours after injury. Leptin also peaks in the circulation 12 hours after wounding. These results suggest that topical treatment with leptin accelerates the healing process.

The present invention is further based on the inventor's study of the pharmacological action of leptin to promote and/or accelerate wound repair in normal animals. The inventor developed a novel, quantitative micromorphometric analysis method that allows a comprehensive and systematic evaluation of wound repair in a murine model of full-thickness incision wounds. This method provides an unambiguous set of morphometric indices involving specific distances and areas measured across the wound bed in a histological section obtained from the geometrical center of the incision. By utilizing these quantitative parameters, the inventor demonstrated that the topical use of exogenous leptin significantly increases the degree of contraction while decreasing epithelial gap length and amount of granulation tissue, thereby reducing the overall area of the wound. Furthermore, increased content of α-SMA mRNA and protein is observed after 5 days of leptin treatment, suggesting regulation of wound contractility. Leptin treatment also alters the cellular abundance of transcripts for collagens I, III and IV, and it markedly accelerates maturation of collagen fibers. While not wishing to be bound by any particulary theory, it is believed that direct topical application of leptin onto wounds modulates local expression of critical effector molecules that mediate key events in wound healing. These findings demonstrate that leptin exhibits features of a potent wound healing-promoting cytokine, which is believed to be of considerable therapeutic value for the treatment of both acute and chronic wounds, both internal and external.

The evaluation of the pharmacological effects of an agent on the dynamic process of wound healing ideally requires a systematic, reproducible and quantitative approach that measures specific structural parameters characteristic of wound tissue. Gross macroscopic measurements of wounds are highly variable and the extent of tissue repair is difficult to quantify as scab material can mask the existing status of the regenerating skin beneath the surface. The micromorphometry method described in the Examples combines a murine model of full-thickness bilateral incisions, single cytokine application on the fresh wound bed, a 72-hour endpoint and a micromorphometric image analysis of the wound bed, focusing on relevant parameters to assess healing progression. Incision wounds of a predetermined uniform size are technically easy to perform at an anatomical location on experimental animals. The single treatment immediately after wounding ensures consistent delivery of the pharmacological agent. Thus, a one-time topical administration avoids potentially confounding factors due to repeated treatment applications, which may alter the wound anatomy and could exhibit variable degrees of bioavailability due to differences in permeability or composition of the natural wound fluid. The endpoint of 72 hours was chosen because at that time, untreated wounds are not fully healed and therefore have discernible elements that characterize the wound bed. Consequently, effects on the early stages of healing by putative wound healing-promoting agents can be assessed more accurately. Wound tissue collection and transversal bisection of the wound tissue flap after euthanasia is straightforward, and standard histological processing/capturing of digital images is readily available in almost any research environment. In addition, when predetermined parameters are measured, computer-assisted morphometry is consistently reproducible when performed by independent observers. Furthermore, similar scores are obtained through a less objective but more typical histopathological assessment performed by a trained dermatopathologist.

Evaluation of digital images obtained from histological sections of regenerating wounds revealed that leptin treatment significantly reduced the relative abundance of granulation tissue and overall wound area, while enhancing contraction and re-epithelialization. In addition, leptin treatment of wounds also increased gene and protein expression of α-SMA and collagens I, III and IV. The results of the micromorphometric and molecular analyses described herein suggest that leptin-treated wounds undergo rapid closure, have reduced overall wound bed areas and exhibit structural characteristics indicative of successful healing progression.

Compositions Comprising Agents According to the Present Invention that Modulate Angiogenesis

In the treatment of the clinical conditions noted above, the compounds of this invention may be utilized in compositions such as tablets, capsules or elixirs for oral administration, suppositories for rectal administration, sterile solutions or suspensions for parenteral or intramuscular administration and the like.

The inventive therapeutics may be administered by any appropriate technique, as will be readily appreciated by those of skill in the art.

In various embodiments, the leptin and/or leptin receptor in the inventive therapeutics may be derived from any natural or synthetic source. Examples include but are not limited to, human, rodent, bovine, avian, production by recombinant expression of nucleic acid molecules encoding the leptin and/or leptin receptor in a suitable host.

In further embodiments, the present invention includes compounds that affect the leptin receptor to promote and/or accelerate wound repair, re-epithelialization, wound contraction, and decrease granulation tissue.

In various embodiments of the present invention, the composition may include additional active ingredients to promote and/or accelerate wound repair.

In various embodiments, the present invention provides pharmaceutical compositions including a pharmaceutically acceptable excipient along with a therapeutically effective amount of leptin. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Antibodies to Leptin, Leptin Receptors, Polypeptide Fragments Thereof

Another embodiment of this invention relates to creating antibodies and antibody fragments that modulate leptin and/or leptin receptor activity and the interaction between leptin and the leptin receptor.

An “epitope” refers generally to a specific recognition feature of a molecule, which depends on the topological orientation of functional groups of the molecule.

According to the invention, a molecule contains an epitope, or shares an epitope of a second molecule, if the first molecule specifically binds or interacts competitively with the specific binding of the second molecule. There is no requirement that shared epitopes be chemically identical; however, shared epitopes must be topologically similar (i.e., have a topological arrangement of chemical functional groups that is similar in each molecule), in order to interact competitively with a target molecule. In another of its embodiments, the present invention relates to antibodies that target or bind to one or to more than one epitope on either leptin or the leptin receptor.

By “antibody” is meant a polyclonal or monoclonal antibody which is capable of binding to leptin, the leptin receptor, or a leptin receptor ligand and modulating thereby their angiogenic, wound healing and/or repair of ischemic tissue activity. Such antibodies can recognize three dimensional regions of these proteins or may be anti-peptide peptides. The term “antibody” therefore encompasses monoclonal and polyclonal antibodies and fragments thereof (e.g., Fv, scFv, Fab, Fab′, or F (ab′)2 fragments). The antibodies contemplated also include different isotypes and isotype subclasses (e.g., IgG, IgG2, IgM, to name a few). These antibodies can be prepared by raising them in vertebrates, in hybridoma cell lines or other cell lines, or by recombinant means. Also contemplated are chimeric, human, and humanized antibodies and fragments thereof, which will be less immunogenic in the subject in which they are administered (e.g., a human or humanized antibody administered to a human subject).

For references on how to prepare these antibodies, see D. Lane, Antibodies: A Laboratory Manual (Cold Spring Harbor Press, Cold Spring Harbor N.Y., 1988); Kohler and Milstein, (1976) Eur. J. Immunol. 6: 511; Queen et al. U.S. Pat. No. 5,585,089; and Riechmann et al., Nature 332: 323 (1988).

Sequences comprising domains on leptin, the leptin receptor or leptin receptor ligands which are immunogenic for purposes of creating antibodies can be determined using such algorithms as described by Hopp and Woods, Proc. Nat'l Acad. Sci. USA 78: 3824 (1981); and Garnier et al., J. Mol. Bio. 120: 97 (1978). Additional algorithms would be known to the skilled artisan and can be used to identify peptides suitable for anti-peptide antibody production.

Combination Therapy

Use of leptin and/or leptin receptor proteins, the nucleic acid molecules encoding them or agents that modulate their expression in combination with other angiogenic or anti-angiogenic factors is also contemplated. The agents to be administered in combination with leptin or other agents that modulate leptin or leptin receptor activity include, but are not limited to, those agents described in: N. Catsimpoolas et al., (1988) U.S. Pat. No. 4,778,787; D'Amato (1998), G. S. Schultz et al., (1991) Eye 5: 170; B. M. Spiegelman et al., (1992) U.S. Pat. No. 5,137,734 (angiogenic monoglycerides); T. Maione (1992) U.S. Pat. No. 5,112,946; C—H. Heldin et al., (1993) U.S. Pat. No. 5,227,302; R. B. Whitman et al., (1995) U.S. Pat. No. 5,470,831; Parish (1997); H. App et al., (1998); P. Bohlen et al., (1997) U.S. Pat. No. 5,641,743; Maione et al., (1992); and D. H. Carney et al., (1996) U.S. Pat. No. 5,500,412.

Agents of the present invention that modulate the activity of leptin and/or leptin receptor can be provided alone, or in combination with other agents that modulate a particular biological or pathological process. For example, leptin can be administered in combination with VEGF (or PDGF and FGFs, TNFa, IL-1 IL-11 or IL-6) to enhance angiogenesis. The examples of combination therapy provided below are specific to regulation of leptin and/or leptin receptor activity. Other combination therapies involving leptin and leptin receptor ligands are also contemplated in the present invention. The therapies described by enhanced angiogenesis spurred by leptin being only one example.

As used herein, two agents are said to be administered in combination when the two agents are administered simultaneously or are administered independently in a fashion such that the agents will act at the same time. Other embodiments include the administration of two or more agents that regulate leptin receptor activity, leptin activity, or both. One illustration includes combinations of agents wherein two or more leptin or leptin receptor antagonists or two or more agonists are administered to a subject.

Typical dosages of an effective leptin or leptin receptor agonists or antagonists can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

Kits

The present invention is also directed to a kit to promote and/or accelerate wound repair, re-epithelialization, wound contraction, and decrease the amount of granulation tissue. The kit is useful for practicing the inventive method of treating wounds. The kit is an assemblage of materials or components, including at least one of the inventive compositions. Thus, in some embodiments the kit contains a composition including leptin, as described above.

The exact nature of the components configured in the inventive kit depends on its intended purpose. For example, some embodiments are configured for the purpose of treating vertebrate specie subjects with wounds. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to promote, enhance, and/or accelerate wound repair. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in wound treatment systems. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing leptin. The packaging material generally has an external label which indicates the contents and/or purpose of the kit and/or its components.

Methods for Quantitative Micromophometric Analysis for the Study and Evaluation of Wound Repair

Other embodiments of this invention include methods for the study and evaluation of wound repair by quantitative micromorphometric analysis of the wounds as described in the examples herein.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Preparation of Animals

Protocols involving mice experiments were first reviewed and approved by the Yale and Cedars-Sinai Animal Care and Use Committees, observing all appropriate institutional guidelines. Female C57BL/6J mice (Jackson Laboratories, Bar Harbor, Me.) were used between 6-8 weeks of age. After wounding procedures, the mice were singly housed in microisolator cages.

### Example 2

### Conducting of Wounding Procedure, Treatment and Collection of Wound Tissue

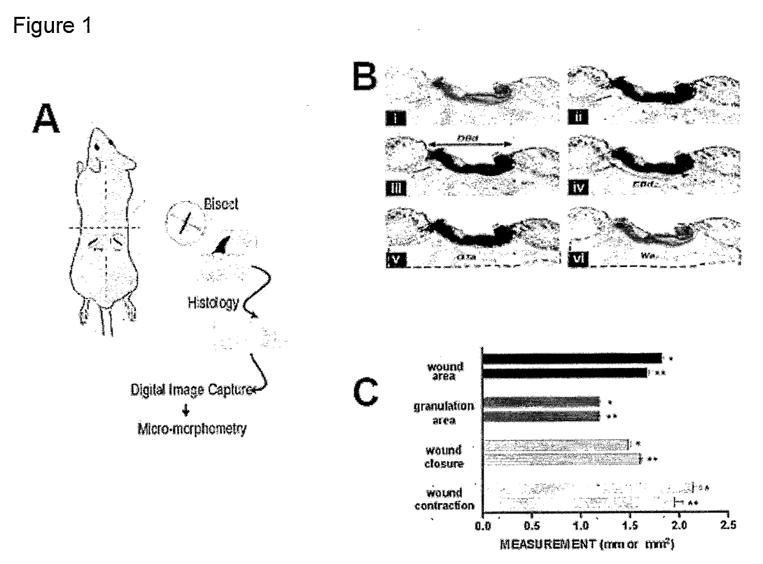

The animals were anesthetized with ketamine (10 mg/kg, i.m.) and Xylazine (40 mg/kg, i.p.). After shaving and disinfecting the skin with 70% ethanol, an 8-mm line was traced on each side on the mid-dorsal region with a surgical skin marker (see FIG. 1A). The skin was firmly retracted and bilateral full thickness dermal wounds were created using fine surgical scissors. The panniculus carnosum was always cut but care was taken not to damage the abdominal wall. Preliminary leptin dose-response experiments were performed using a dose range of 0.1-50 μg leptin/wound (Calbiochem, La Jolla, Calif.). In subsequent experiments, wounds of each mouse received a topical treatment with a pre-established optimal dose of leptin (10 μg/wound) or saline in a volume of 15 μl (n=22). Time points of 24-96 hours were evaluated by morphometric analysis. Wound borders were not mechanically juxtaposed and no dressing was applied on the wounds. Wounds were examined at the indicated times. After euthanasia, the histological samples for analysis were obtained from a tissue flap that comprised the entire wound bed and underlying tissues, including the dorsal muscular layer. The samples were carefully bisected at the geometric center of the incision line. Cross-section specimens were fixed overnight in buffered formalin (Sigma, St. Louis, Mo.) and embedded in paraffin for sectioning. Hematoxylin and eosin (H&E) staining was routinely preformed on 4 μm sections. Excision wounds were used for gene experiment analysis and for macroscopic evaluation. Two excision wounds were created per mouse using a disposable 3-mm biopsy punch (Biopunch, Fray Corp., Amherst, N.Y.). Excision wounds were collected at the indicated times after euthanasia using a 6-mm biopsy punch. The tissue specimens were soaked in RNALater (Ambion, Austin, Tex.) and stored at 4° C. for a maximum of one week, until processed for RNA extraction.

### Example 3

### Macroscopic, Micromorphometric and Histopathological Analysis

Macroscopic images of wounds were captured using an Olympus Camedia Digital Camera C-3040ZOOM with an Olympus Super Bright Zoom Lens (7.1-21.3 mm Lens) (Olympus Corporation, Japan). For micromorphometric analysis, H&E slides were randomly coded and digital images were acquired for analysis with an IPLab Spectrum v. 3.2.4 digital microscopy software program (Scanalytics Fairfax, Va.; see FIG. 1A). An image obtained from a graduated stage micrometer was used to calibrate the imaging software for automatic conversion of pixel units to millimeters. To evaluate wound contraction, the distance between dermis borders was measured by tracing a straight-line between the normal dermis tissues on each side of the wound (DBd; FIG. 1B, iii). To assess wound closure, re-epithelialization was measured as the length between the migrating epithelial tongues along the surface of the unhealed wound (EBd; FIG. 1B, iv). Granulation tissue content was measured by digitally enclosing the granulation tissue discernible by histology inspection (GTa; FIG. 1B, v). Wound area was measured by visually discriminating normal and wound tissue and enclosing the area encompassed by all of the morphological elements of the wound (Wa; FIG. 1B, vi). To validate the computer-assisted morphomery, the slides were also scored blindly by a trained clinical dermatopathologist (C.C.). R^{e}-epithelialization was measured using an ocular micrometer installed in the eyepiece of the microscope. Granulation tissue was scored on the following semi-quantitative scale: 1, not present or minimally present; 2, low density; 3, moderate density; and 4, high density (see Table 1).

TABLE I
Dermatopathological Evaluation of
Leptin- and Saline-treated Wounds*

| Parameter Measured | Saline | Leptin |
|---|---|---|
| Re-epithelialization (mm) | 1.34 ± 0.18 | 0.55 ± 0.14 |
| Granulation Tissue | 2.00 ± 0.26 | 1.20 ± 0.13 |

*Scoring scale for H&E-stained slides was: 1, thin; 2, moderate; 3, thick; n = 22 for each group; Values are average ± S.E.M. Comparison between groups was done using a Student's t test. Microscopic evaluation and scoring was performed by a board-certified dermatopathologist blinded to the experiment.

### Example 4

### Histology and Immunohistochemistry (IHC)

Paraffin-embedded 4 μm sections of bisected wounds were routinely stained with H&E (Mass Histology Service, Warwick, R.I.). Histochemistry was routinely performed on 10 μm frozen sections. Immunohistochemistry for α-SMA was carried out using an alkaline phosphatase-conjugated monoclonal antibody (Sigma, St. Charles, Mo.), and processed using an ABC kit (Vector Labs) for signal amplification and Vector Alkaline Phosphate Substrate kit for detection. Phosphomolybdic acid-modified picrosirius red (PMA-PSR) stain was used to visualize collagen fibers in paraffin sections (Dolber PC, Spach MS).

### Example 5

### RNA Extraction and Quantitative RT-PCR

Total RNA was isolated from mouse skin samples by using two consecutive extractions with Trizol® (Invitrogen, Carsbald, Calif.) to ensure RNA purity. Before cDNA synthesis, the samples were digested with DNase I to eliminate any residual genomic DNA contamination (Ambion, Austin, Tex.). cDNA synthesis was performed using SuperScript™ II (Invitrogen, Carlsbad, Calif.) and HotStarTaq™ DNA polymerase (Qiagen, Valencia Calif.) was then used for PCR amplification reactions. Quantitative PCR (qPCR) amplicon detection was achieved using a Biorad iCycler iQ real-time PCR cycler in combination with 5′FAM/3′ BHQ-1 dual-labeled fluorogenic Taqman® probes (Biosearch Technologies, Novato, Calif.), flanked by appropriate forward (fwd) and reverse (rev) primers.

### Example 6

### Statistical Analysis

Results are expressed as mean values ±standard error. Data were analyzed by two-tailed Student's t test using the InStat3 software program (GraphPad Software, Inc. San Diego, Calif.). Differences considered to reach statistical significance had probability values less than or equal to 0.01.

### Example 7

### Histological Appearance of the Wounds

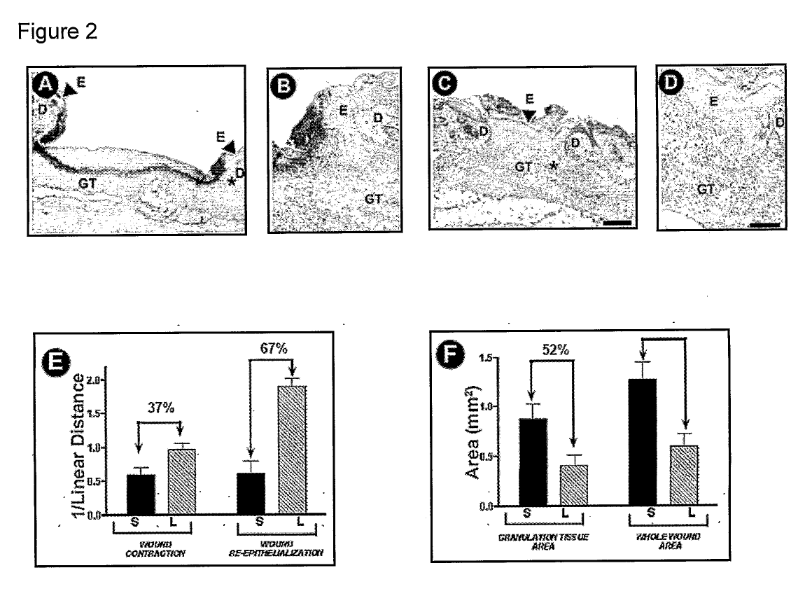

Leptin treatment was performed immediately after the wounding procedure by directly applying onto the wound an adequate pharmacological dose of leptin, which had been previously determined in initial experiments. A representative example of the microscopic appearance of a control and a leptin-treated wound is shown in FIG. 2. After 3 days, the control wound exhibited substantial granulation tissue content and the epithelial layer had undergone partial regeneration covering approximately one third of the wound underneath the occluding scab. The control wound also had a significant level of inflammatory infiltrate characteristic of uncomplicated healing of the skin barrier. However, there was no evidence of basement membrane formation across the wound, which was only moderately contracted (FIGS. 2A and B). In contrast, the leptin-treated wound had achieved complete re-epithelialization, exhibiting a well-defined basement membrane and it appeared fully contracted. In addition, the wound was fully closed with only a moderate amount of inflammatory infiltrate present. Finally, the granulation tissue had already begun to recede and in the process of being replaced by connective tissue fibers to ultimately form the mature scar (FIGS. 2C and D)

### Example 8

### Micromorphometric Assessment

At the onset and on a macroscopic scale, the overall size of the wou nds treated with leptin generally appeared to be smaller. However, incision wounds normally tend to rapidly contract and close, with the scab often concealing the undergoing regenerative process beneath it. For this reason, no macroscopic assessment was attempted. Instead, all measurements were conducted using low magnification (25×) digital micrographs obtained from 4 μm H&E-stained tissue sections. Four specific morphometric parameters were systematically measured in control and leptin-treated wounds: a) wound contraction; b) re-epithelialization or wound closure; c) granulation tissue abundance; and d) overall wound tissue area. To assess accuracy in the data collected for the morphometric analysis, two independent investigators were instructed on the general definition of each parameter and trained to use the imaging software. They were then given sixty digital images of experimental wounds and asked to examine them independently. The results of their recorded measurements are shown in FIG. 1C. In all four parameters measured, the difference between the values determined by each independent investigator ranged collectively from 0.1% to 8.4%. Granulation tissue area measurements exhibited the least variability, whereas the distance between dermal borders showed the greatest difference. Nonetheless, these differences were confined to a variation range that never exceeded 10% of the mean value, which we regard as a reasonable level of experimental error. These results serve to validate the overall consistency in the micromorphometry parameters of the method (regardless of the individual performing the measurements), thereby conferring a rational basis for an objective quantitative method to assess wound healing.

### Example 9

### Wound Contraction

Contraction is an important event during wound repair arising from the contractile activity of myofibroblasts, which are normal cellular elements of the provisional matrix. Contraction begins early and serves to close the gap between uninjured borders. The mechanical juxtaposition of the borders minimizes exposure to the environment, hence preventing fluid loss and reducing the amount of tissue to be regenerated. The morphometry results show that leptin treatment caused a 37% increase in contraction when compared to saline controls (FIG. 2E). Contraction was measured as the inverse value of the linear distance between dermis borders. Thus, it appears that leptin significantly enhances wound contraction by reducing the inter-dermal border distance. Although this effect could reflect modulation of discrete events such as recruitment of fibrocytes to the injured site, their differentiation into myofibroblasts within the wound bed or changes in their contractile function, the precise mechanism by which leptin increases wound contraction remains to be elucidated. However, in this regard, the inventor has recently demonstrated that expression of the leptin gene occurs in dermal fibroblasts and is rapidly induced in response to hypoxia, an effect that is mediated by activation of hypoxia inducible factor-1 (Ambrosini et al., 2002. Transcriptional activation of the human leptin gene in response to hypoxia: Involvement of hypoxia-inducible factor 1. J. Biol. Chem.). In addition, cultured fibroblasts also express functional leptin receptors, including the signaling competent long form of the leptin receptor (OB-Rb) (Glasow et al., 2001. Expression of leptin (Ob) and leptin receptor (OB-R) in human fibroblasts: regulation of leptin secretion by insulin. J Clin Endocrinol Metab 86, 4472-4479.) Thus, leptin may exhibit important autocrine and paracrine effects during the early phases of the tissue regeneration process within the wound bed.

### Example 10

### Re-Epithelialization

The process of re-epithelialization begins with keratinocyte proliferation and migration. The denuded surface of the wounded skin undergoes a rapid initial resurfacing by a monolayer of epithelium. Then, proliferating epithelial borders gradually advance to regenerate the skin surface. Although some of the signals for these two key processes are known to be mediated through cytokines such as KGF-1, KGF-2, EGF and TGF-α, it has recently been shown that leptin also induces keratinocyte proliferation and enhances migration of the epithelial tongues in experimental wounds (Frank et al., 2000. Leptin enhances wound re-epithelialization and constitutes a direct function of leptin in skin repair. J Clin Invest 106, 501-509). The inventor's findings using quantitative micromorphometry show that leptin treatment markedly promotes re-epithelialization, as measured by the inverse value of the linear distance between advancing epithelial tongues (FIG. 2E). Specifically, wounds treated with leptin were 67% more re-epithelialized than saline-treated control wounds (p<0.01).

### Example 11

### Granulation Tissue Area

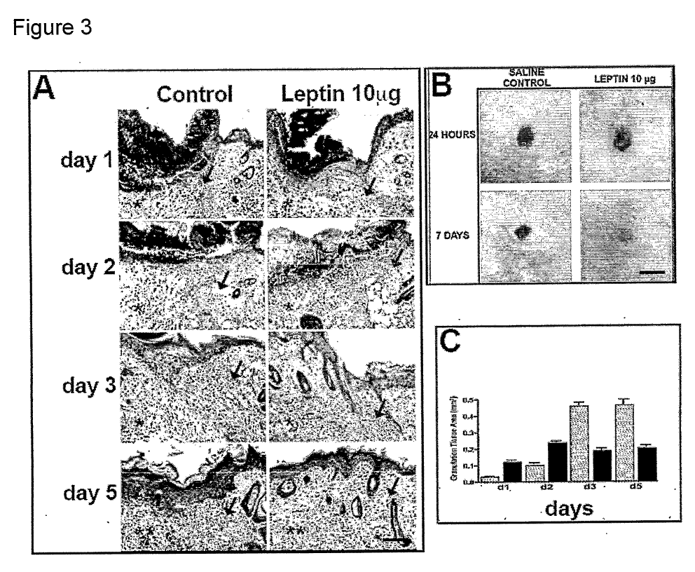

During the inflammation phase of wound healing, platelets and macrophages release cytokines and growth factors that initiate the formation of granulation tissue, which consists primarily of provisional matrix and newly formed blood vessels. As healing progresses, this granulation tissue gradually reabsorbs and is substituted by the scarring matrix regenerating the dermis. Leptin treatment of wounds caused a 53% reduction in granulation tissue abundance when compared to controls (FIG. 2F). These findings suggest that treatment with exogenous leptin may diminish the overall formation of granulation tissue or provisional matrix. However, it is likely that the observed reduction in granulation tissue content is simply the result of leptin-induced contraction of the wound, which narrows the tissue gap between epithelial borders thus limiting the physical area available for deposition of granulation tissue. To determine if the apparent deficit of granulation tissue was directly related to leptin treatment (and not simply a reflection of a more advanced stage of healing), its abundance in control and leptin-treated wounds was quantified at various times. As shown in FIG. 3A, the presence of granulation tissue in leptin-treated wounds is already apparent in tissue sections at 24 hours, but not in the saline control, in which it is only incipient [0.126±0.033 vs. 0.0350±0.013, respectively (n=16)]. Notably, granulation tissue content in leptin-treated wounds reaches a peak 24 hours earlier than control wounds (FIG. 3C), albeit its production is approximately one-half of that observed in saline-treated control wounds. These data demonstrate that granulation tissue appears earlier in leptin-treated wounds and suggest that the reduction in maximal granulation tissue content observed at 72 hours is probably the result of smaller whole wound size (see below).

### Example 12

### Wound Area

The overall wound area consistently included the epithelial borders, provisional matrix, granulation tissue and scab tissue. In accordance with the changes observed in the other parameters measured, leptin treatment significantly diminished overall wound area when compared to control wounds. Specifically, there was a 53% reduction of wound area in leptin-treated wounds (see FIG. 2F).

### Example 13

### Time Course

To define the evolution of the morphometry parameters previously described, wound sections were studied and compared at various times (see FIG. 3). At 72 hours, healing activity in the wound was fully organized and had normally progressed to a stage where the morphometric parameters were clearly measurable. Thus, 72 hours was selected as the most suitable time at which to collect morphometric observations. However, to evaluate qualitative microscopic changes in wound healing due to leptin treatment, H&E sections were studied at various times as well (FIG. 3). It is evident that leptin-treated wounds exhibited much earlier the typical features of control wounds that are normally observed at later time points (48 and 72 hours), including discernible granulation tissue, epithelial tongue regeneration, wound contraction and dense provisional matrix (FIG. 3A). After five days, both control and leptin-treated wounds were fully closed. However, while the control scar tissue visibly contained more cellular inflammatory infiltrate and vascular elements, the leptin-treated scar showed a more quiescent appearance consistent with a late remodeling stage (FIG. 3A).

Macroscopically, the wounds treated with leptin had apparent smaller diameters (FIG. 3B). However, during the first 3 days after wounding, the macroscopic appearance of the wounds showed considerable variability. As a result, the macroscopic morphometry did not yield statistically significant differences between control and leptin-treated wounds (not shown). In general, macroscopic measurements during the early phases of healing could be misleading as the scab film completely covers the underlying tissue, thereby concealing the actual degree of healing progression. This is best illustrated in FIG. 3, in which different degrees of epithelialization are apparent despite the scab tissue covering the wound. Macroscopically, while control untreated wounds are still covered by scab tissue after 7 days, leptin-treated wounds appear completely healed (FIG. 3B).

### Example 14

### Dose-Response

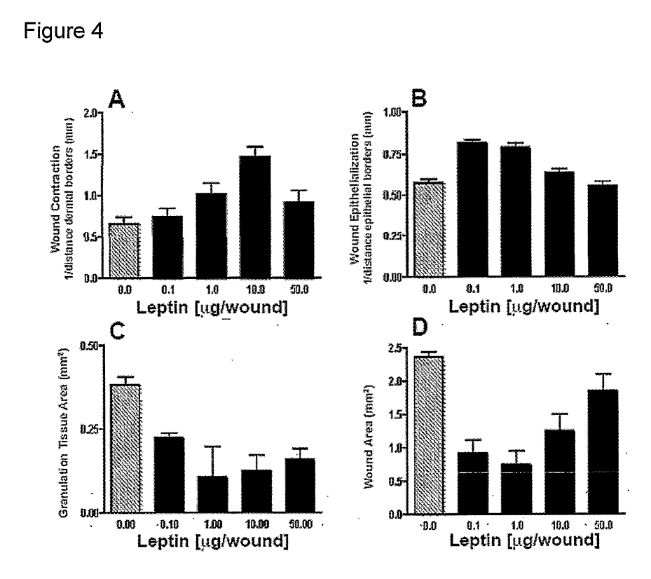

Dose-response experiments were performed by administering 0.1, 1, 10 or 50 μg of leptin as single topical applications to find the optimal dose for treatment of incision experimental wounds in mice. Each dose was applied at the time of wounding for 72 hours, followed by euthanasia, wound collection and histological evaluation. Micromorphometric analysis was performed using the parameters previously described (see FIG. 1). As shown in FIG. 4A, it is evident that leptin markedly increases (by 2-fold) wound contraction in a dose-dependent fashion, with a maximal effect observed with 10 μg of leptin. In addition, the degree of epithelialization was significantly higher in leptin-treated wounds, although in this case a maximal effect was achieved at lower doses (0.1 and 1 μg; FIG. 4B). Likewise, leptin significantly reduced wound and granulation tissue areas, with a maximal effect observed at the 1 μg dose (FIGS. 4C and 4D). Taken together, these results indicate that leptin is effective in reducing overall wound size and accelerating wound closure; the smaller tissue gap consequently required less granulation tissue. Although most doses used seemed to have an impact on the parameters examined, lower submicrogram doses produced more discernible effects on epithelialization and granulation tissue abundance. The behavior of most of the parameters measured in dose-response experiments were consistent with a “bell-shaped” response curve with a diminished response at the highest dose used (50 ug). This phenomenon is typically seen at pharmacological concentrations of bivalent ligands, when bound ligand molecules fail to adjoin a second receptor due to occupancy.

### Example 15

### Dermatopathology Assessment

In order to determine the reliability and accuracy of the computer-assisted method described herein, the same H&E slides of leptin and control treated wounds that were submitted to micromorphometry were also independently evaluated by a trained dermatopathologist. Parameters scored by microscopic observation were re-epithelialization and granulation tissue. Values recorded for all parameters reached statistical significance (p<0.01) and comparison to the computer-generated measurements revealed that our model of analysis produced similar results (Table 1). For example, the dermatopathologist scored leptin-treated wounds as 61% more re-epithelialized compared to saline, while the analysis by micromorphometry in our hands revealed a 68% increase. Likewise, the dermatopathologist scored leptin-treated wounds as containing 36% less granulation tissue, whereas wound morphometry showed a 53% decrease.

### Example 16

### Expression of α-SMA

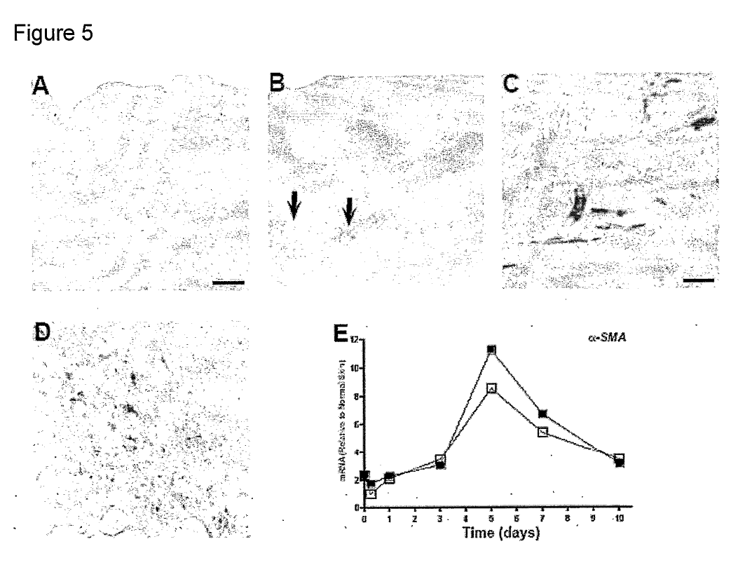

Expression of α-SMA was evaluated to assess whether increased contraction and reduced wound area caused by leptin treatment could be explained by increased content of myofibroblasts. Leptin-treated wounds (10 μg/wound) displayed enhanced α-SMA immunoreactivity in fibroblasts present in the wound bed by day 5, but not in untreated wounds (FIG. 5A through D). Furthermore, a time course of α-SMA mRNA accumulation followed over a 10 day period after wounding revealed a peak level on day 5 (FIG. 5E). Of note, the tissue content of α-SMA mRNA was higher in leptin-treated wounds than that in untreated controls (12-vs.8-fold, respectively).

### Example 17

### Expression and Distribution of Collagens I, III and IV

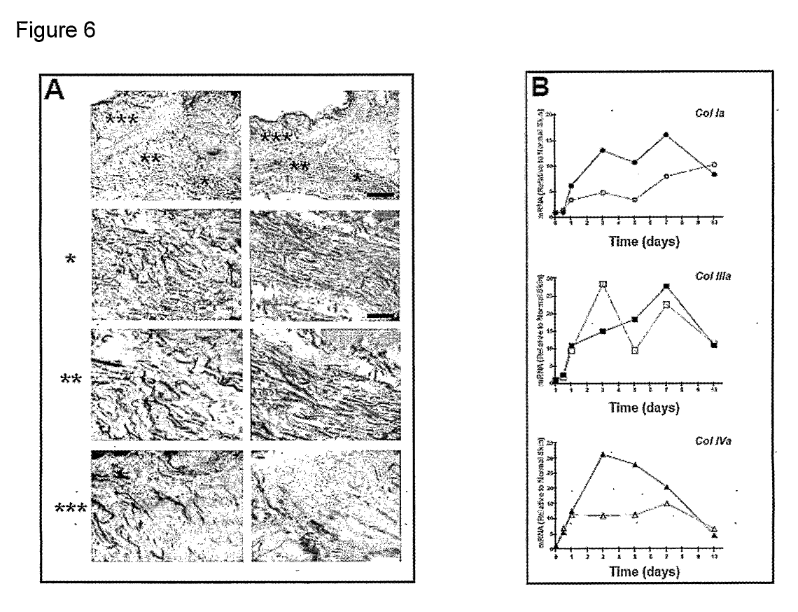

The earliest collagen fibrils in the scarring dermis are mainly composed of short and coiled type III collagen, which are subsequently replaced by long, straight and highly organized type I collagen fibrils (Robins, S P et al.,). To evaluate if leptin treatment might have an impact on matrix deposition and scar tissue formation, histological sections of 5-day wounds were stained using a modified Picrosirius Red method. This method allows the visualization of collagen fibers by fluorescence microscopy without the confounding effects of cytoplasmic fluorescence (polber et al. (1993) H. Histochem. Cytochem. 41: 465). The collagen fibers thus visualized in several regions of the developing scar showed significant differences in the length, density and organization between leptin-treated and control wounds (FIG. 6A). Whereas leptin-treated wounds displayed longer, well-organized and more abundant fibers, control wounds contained fewer fibrils with a short and coiled appearance when inspected at higher magnification. Accompanying these morphological findings, mRNA expression experiments performed in parallel showed that leptin treatment induced an early, rapid and sustained increase in procollagen α1(I) mRNA levels (FIG. 6B). Conversely and as expected, the content of procollagen α1 (III) mRNA increased in control wounds, exhibiting a biphasic pattern of expression with two distinctive peaks occurring at days 3 (30-fold increase) and 7 (22-fold increase) after wounding (FIG. 6B). However, leptin treatment markedly obliterated the appearance of the first early procollagen α1(III) mRNA peak and slightly enhanced the magnitude of the second peak observed at day 7, compared to the control (27-vs. 22-fold). These results are consistent with the aforementioned morphological difference in the apparent deposition of collagen fibrils observed in wounds at day 5 (FIG. 6A).

Type IV collagen is the major collagen present in basement membranes. During healing, basement membranes of the epidermal epithelium and vascular endothelium are regenerated and therefore require de novo synthesis of type IV collagen. Thus, expression of procollagen α1(IV) mRNA in control untreated wounds reached a plateau on day 1, which remained steady at least until day 7. In contrast, leptin-treated wounds exhibited a rapid increase peaking on day 3. The magnitude of this leptin-mediated induction was much higher (30-fold) than control wounds (FIG. 6B). These findings strongly suggest an accelerated regeneration of basement membranes to allow rapid and proper reepithelialization of the skin. Basement membrane is also essential for the maturation of newly formed vessels in the wound bed. These findings are therefore consistent with rapid progression of wound healing induced by leptin.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method of promoting and/or accelerating wound contraction in a subject, comprising:
providing a composition comprising an agent that induces a leptin or leptin receptor-mediated response; and
administering a therapeutically effective amount of the composition to the subject.

2. The method of claim 1, wherein the agent is leptin.

3. The method of claim 1, wherein the subject is a mammal.

4. The method of claim 1, wherein administering the therapeutically effective amount of the composition to the subject further comprises:
administering the therapeutically effective amount of the composition to a wound.

5. The method of claim 1, wherein the composition further comprises:
a pharmaceutically acceptable carrier.

6. The method of claim 1, wherein administering the therapeutically effective amount of the composition is performed via a route of administration selected from the group consisting of aerosol, nasal, oral, transmucosal, transdermal, parenteral and combinations thereof.

7. A method of promoting and/or accelerating wound repair in a subject, comprising:
providing a composition comprising an agent that induces a leptin or leptin receptor-mediated response; and
administering a therapeutically effective amount of the composition to the subject.

8. The method of claim 7, wherein the agent is leptin.

9. A method of promoting and/or accelerating re-epithelialization in a subject, comprising:
providing a composition comprising an agent that induces a leptin or leptin receptor-mediated response; and
administering a therapeutically effective amount of the composition to the subject.

10. The method of claim 9, wherein the agent is leptin.

11. A method of decreasing an amount of granulation tissue, comprising:
providing a composition comprising an agent that induces a leptin or leptin receptor-mediated response; and
administering a therapeutically effective amount of the composition to the subject.

12. The method of claim 11, wherein the agent is leptin.

13. A wound repair composition, comprising:
a quantity of an agent that induces a leptin or leptin receptor-mediated response; and
a pharmaceutically acceptable carrier.

14. The wound repair composition of claim 13, wherein the agent is leptin.

15. A kit, comprising:
a wound repair composition, comprising:
a quantity of an agent that induces a leptin or leptin receptor-mediated response, and
a pharmaceutically acceptable carrier; and
instructions for the use of the wound repair composition to promote and/or accelerate wound repair.

16. The kit of claim 15, wherein the agent is leptin.

17. A method for studying wound healing, comprising:
obtaining a tissue flap that comprises an entire wound bed and underlying tissues samples of a wound;
bisecting the samples at the geometric center of the incision line;
performing hematoxylin and eosin (H&E) staining;
acquiring a digital image a of H&E slide;
obtaining an image using a micrometer;
calibrating an imaging software for automatic conversion of pixel units to millimeters by using the image obtained from the micrometer; and
performing a measurement of an aspect of the wound bed selected from a group consisting of wound contraction, re-epithelialization, granulation tissue content and combinations thereof.
